(19) United States
(12) Patent Application Publication
(10) Pub. No.: US 2012/0160241 A1
(43) Pub. Date: Jun. 28, 2012

(54) UPGRADED INHALATOR FOR POWDER PREPARATIONS

(76) Inventor: Roberto Oliva, Carpi (IT)

(21) Appl. No.: 13/394,402

(22) PCT Filed: Jun. 14, 2010
(86) PCT No.: PCT/IB10/01432

§ 371 Date: Mar. 6, 2012

(30) Foreign Application Priority Data
Sep. 30, 2009 (IT) MO2009A000240

(51) Int. Cl. A61M 15/00

(52) U.S. Cl. 128/203.15

(57) ABSTRACT

An upgraded inhalator for powder preparations comprises a main body having an inhalation channel, an external opening accessible by a user, and a tank adapted to receive a dose of powder preparation. The tank is opened to establish communication with the inhalation channel. At least one protuberance protrudes or extends towards the inside of the inhalation channel. Each protuberance defines substantially concave impact surface and is arranged on the opposite side to the external opening. The impact surface of each protuberance intercepts the powder preparation during its crossing of the inhalation channel towards the external opening.

This application is a National Stage of PCT International Patent Application No. PCT/IB2010/001432, filed Jun. 14, 2010, and claims priority to Italian Patent Application No. MO2009A000240, filed Sep. 30, 2009, in the Italian Patent Office, the disclosures of which are incorporated herein by reference.

### BACKGROUND OF THE INVENTION

1. Field of the Invention

The present invention relates to an upgraded inhalator for powder preparations.

2. Description of the Related Art

Inhalators for powder preparations are known requiring the use of capsules containing a dose of preparation. The capsules are individually inserted inside the inhalator which, in different ways, breaks the capsule and makes the corresponding dose of powder preparation available for inhalation.

The inhalators of this type are generally composed of a container comprising an inhalation channel put in communication with a housing chamber for housing the capsule containing the medicinal product to be inhaled.

Associated with the housing chamber are means or devices for breaking the capsule, arranged in correspondence to the extremities of the housing chamber. The devices may be composed of two perforators having, respectively, contrast springs and a certain number of aculei which are able to penetrate the capsule, by applying a certain pressure with the fingers, inside the housing chamber itself. The powder is released from the capsule and is inhaled by the user.

The inhalators of this type, however, have numerous drawbacks, tied mainly to the high number of component parts, such as the aculei, the springs and the extremities required to operate the perforators. Some of such components must move relative to the container, and malfunctions and blockages are therefor possible.

Furthermore, the multiple use of the inhalator leads to the bacteriological contamination of the inhalator by saliva or external agents, such as dust, and consequently cleaning operations can become necessary.

Again, these known inhalators require accurate assembly operations to be able to operate correctly, and these operations involve a consequent increase in production costs and therefor make single-use economically inopportune.

Some drawbacks have been overcome by an inhalator presented in the international patent application WO 2004/035121 filed on behalf of the same applicant. Such inhalator envisages the presence of a main body, having an inhalation channel, secured in a rotatable manner to a secondary body having a housing into which the capsules containing the medicinal product to be inhaled are inserted.

The secondary body can be rotated between an opening position, wherein the housing is accessible from outside for inserting the capsules, and a closing position, wherein the housing is in operative communication with the inhalation channel. This type of inhalator also has cutting means suitable for cutting a portion of the capsule protruding from the housing during the rotation of the secondary body from the opening position towards the closing position, so the contents of the capsule run into the inhalation duct.

This type of inhalator, however more functional and reliable than the inhalators initially described, is not suitable for the use of a loose powder preparation, i.e, not enclosed in a capsule, to be released at the time of use and also has the above drawbacks relating to contamination by saliva and dust.

For this reason, the present applicant has provided a further inhalator for powder preparations, described in the patent application EP 1 725 287.

EP 1 725 287 describes a disposable, single-dose inhalator, composed of a first body, having an inhalation channel, and a second body, having a tank for containing a dose of a powder preparation to be inhaled. The first body is rotated with respect to the second body to allow the powder preparation to fall from the tank into the inside of the inhalation channel.

The rotation of the first body with respect to the second body therefor selectively isolates the tank from the inhalation channel, or establishes communication therebetween.

It is known that the quantities of active medicinal product for lung inhalation are so small, i.e., less than one milligram per dose, that the distribution of single doses can be problematic. For this reason, in most cases, the loose powder preparation to be inhaled also comprises a carrying component (also called “carrier”). The carrier is composed of one or more inert substances made up of particles of larger dimensions compared to those of the active ingredient, and allows the preparation of single doses of active medicinal product in a reproducible way.

However, to ensure that inhalation is efficient, i.e., that the active ingredient is actually inhaled at lung level (i.e., that it reaches the peripheral part of the lung) of the user, the particles containing the active ingredient must be separated from the carrying particles during transit through the inhalation channel.

For this reason, the inhalator described in EP 1 725 287 has protuberances along the inhalation channel, designed to be impacted with the powder preparation during the crossing of the inhalation channel itself towards the outside, i.e., towards the user.

Such protuberances, which perform the function of separating the particles containing the active ingredient from the carrying particles, have a sloped surface turned towards the external opening of the inhalation channel accessible by the user.

This type of inhalator for powder preparations also has a number of drawbacks.

In fact, laboratory tests made on this type of inhalator show that the quantity of the particles of active ingredient freed in the impact with the above-mentioned protuberances and suitable for being absorbed at peripheral lung level (called “breathable fraction”) is lower than that of the powder inhalator devices available on the market.

The quantity of particles freed by the impact with the protuberances in the inhalator described in EP 1 725 287 is not therefore large enough to consider inhalation effective and satisfactory.

Another drawback of the inhalator described in EP 1 725 287 is related to tank loading which is not at all easy and functional.

In fact, as explicitly described in EP 1 725 287, to load the tank, the first body has to be removed from the second body to make the tank accessible from the outside. As it is easy to appreciate, this operation is not at all practical as it requires the disassembly of the inhalator itself.

### SUMMARY OF THE INVENTION

The main object of the present invention is to provide an upgraded inhalator for powder preparations that obtains a breathable fraction of active medicinal product greater than that obtained with the inhalator described in EP 1 725 287.

The present invention provides an inhalator for powder preparations having greater efficiency, i.e., that allows performing more effective inhalation compared to known inhalators, and in particular compared to the inhalator described in EP 1 725 287.

One object of the present invention is to provide an inhalator for powder preparations that allows easier and more functional loading of the tank designed to contain the powder preparation compared to the inhalators of known type.

Another object of the present invention is to provide an upgraded inhalator for powder preparations which overcomes the mentioned drawbacks of the state of the art within the ambit of a simple, rational, easy and effective to use inhalator at a reasonable cost.

The above objects are all achieved by the present upgraded inhalator for powder preparations, comprising a main body having:

an inhalation channel with an external opening accessible by a user;

a tank adapted to contain a dose of powder preparation, the tank being opened establish communication with said inhalation channel;

at least a protuberance protruding towards the inside of said inhalation channel and

characterised by the fact that said protuberance defines at least a substantially concave impact surface and arranged on the opposite side to said external opening, said powder preparation being intercepted by said impact surface during the crossing of, or passage through, said inhalation channel towards said external opening.

### BRIEF DESCRIPTION OF THE DRAWINGS

Other characteristics and advantages of the present invention will become more evident from the description of a preferred, but not sole, embodiment of an upgraded inhalator for powder preparations, illustrated purely as an example but not limited to the annexed drawings in which:

FIG. 1 is a perspective view of the inhalator according to the invention in a preferred embodiment;

FIG. 2 is a longitudinal section view of the inhalator of FIG. 1 with the first body in the first operative position;

FIG. 3 is a longitudinal section view of the inhalator of FIG. 1 with the first body in the second operative position;

FIG. 4 is an exploded perspective view view of the inhalator of FIG. 1;

FIG. 5 is a perspective view of the inhalator according to the invention in an alternative embodiment;

FIG. 6 is a longitudinal section view of the inhalator of FIG. 5 with the first body in the first operative position;

FIG. 7 is a longitudinal section view of the inhalator of FIG. 5 with the first body in the second operative position.

### DETAILED DESCRIPTION OF THE EMBODIMENTS

With reference to such figures, globally indicated by 1 is an upgraded inhalator for powder preparations.

The inhalator 1 comprises a main body 2 having an inhalation channel 3 with an external opening 4 accessible by a user, and a tank 5 designed to contain at least a dose of powder preparation P. The tank is opened to establish communication with the channel 3 and the protuberances 6a, 6b protruding towards the inside of the channel 3.

As is known to experts in the sector, powder preparation refers to a series, or plurality, of particles comprising the particles of the active ingredient to be inhaled and the carrying particles.

According to the invention, protuberances 6a, 6b define a substantially concave impact surfaces 7a, 7b arranged on the opposite sides of the inhalation channel with respect to the external opening 4. The impact surfaces 7a, 7b intercept the powder preparation P during the crossing of the channel 3 towards the external opening 4.

Preferably, the impact surfaces 7a, 7b are curvilinear and are substantially C-shaped or hook-shaped.

The impact surfaces 7a, 7b are shaped to define a tortuous path that must be crossed by the powder preparation P during its movement towards the external opening 4, thereby obtaining a highly turbulent flow of air inside the channel 3 and facilitating the detachment of the particles containing the active ingredient from the carrying particles.

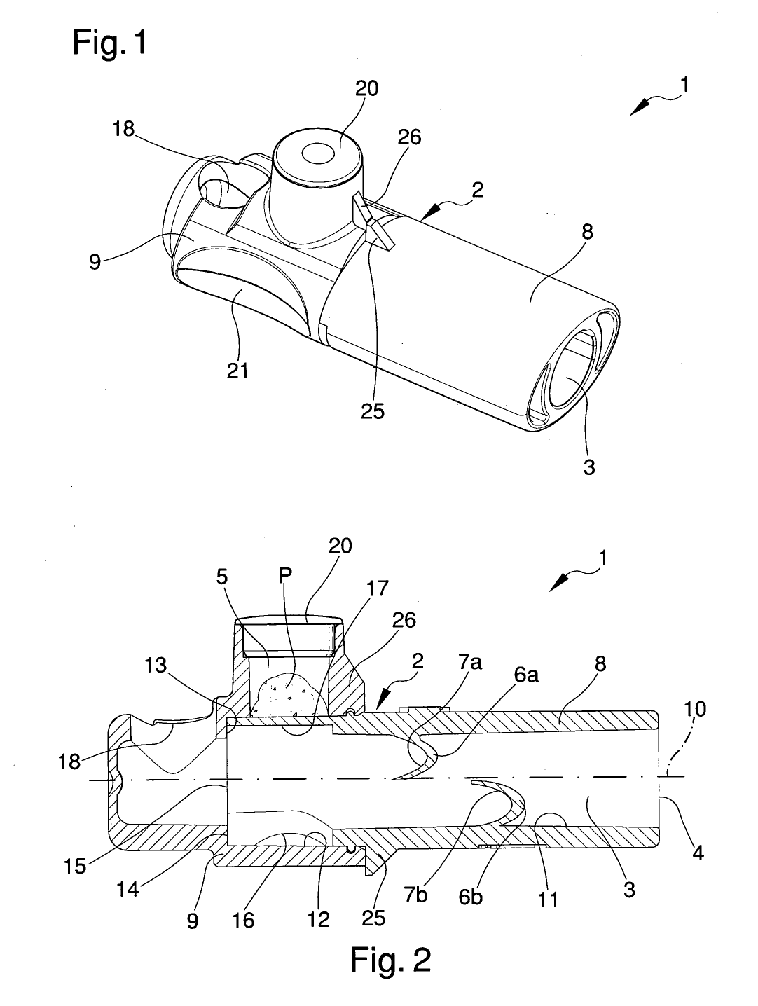

In a preferred embodiment, shown in FIG. 1, the inhalator 1 includes two protuberances 6a and 6b, wherein a first protuberance 6a and a second protuberance 6b define a first impact surface 7a and a second impact surface 7b respectively.

The protuberances 6a and 6b are longitudinally spaced from each other along the channel 3, and are arranged on the opposite sides of a median plane of the inhalation channel itself.

Advantageously, as shown in the embodiment shown in the illustrations, the main body 2 comprises a first body 8, wherein the inhalation channel 3 is defined, and a second body 9, wherein the tank 5 is defined.

Suitably, both the first body 8 and the second body 9 are made of transparent material, so that the user can see from outside the fall of the powder preparation inside the channel 3.

The first body 8 is mobile, i.e. rotatable, with respect to the second body 9 between a first position, wherein it closes the tank 5 isolating it from the channel 3, and a second position, wherein it opens the tank 5, putting it in communication with the channel 3.

Preferably, the first body 8 has an elongated shape and the channel 3 extends along the longitudinal axis of the first body, identified in the illustrations with reference number 10.

More particularly, in the embodiment shown in the illustrations, the first body 8 has a substantially oval outer section and a substantially round internal section defined by the side surface 11 of the channel 3.

The protuberances 6a and 6b protrude from the side surface 11 of the channel 3 crossways to the longitudinal axis 10. The protuberances are spaced relative to each other along the longitudinal axis itself, and are arranged on the opposite sides of the longitudinal median plane of the channel 3.

The second body 9 comprises a housing seat 12 for at least a portion of the first body 8, which can be rotated with respect to the second body 9 inside the housing seat itself.

In the embodiment shown in the illustrations, the first body 8 is rotatable with respect to the second body 9 around the longitudinal axis 10.

The housing seat 12 has a mechanical stop 13 designed to be contacted by a counter-stop 14 defined by the portion of the first body 8 which can be fitted inside the housing seat itself.

The inhalator 1 also includes rotation stop means suitable for indicating to the user that the end of stroke has been reached during the rotation of the first body 8 with respect to the second body 9.

These stop means are made up of a relief and of a recess defining a stop and a counter-stop, respectively, suitable for cooperating when the first body 8 reaches the above-described work or operative positions. More in particular, the stop means are shaped so as to also produce a sound signal when the first body 8 reaches the end-of-stroke position.

Advantageously, the channel 3 is of the through type and has a first port 15, arranged on the opposite side to the external opening 4 with respect to the protuberances 6a and 6b, and a second port 16, defined on the side surface 11 and suitable for putting in communication the tank 5 with the channel 3 in the second position of the above-described first body 8. Suitably, the first and the second port 15 and 16 are defined in the portion of the first body 8 insertable in the housing seat 5 and the second port 16 is placed between the external opening 4 and the first port 15.

In the preferred embodiment shown in FIGS. 1-4, the external opening 4 and the first port 15 are defined in correspondence to the axial extremities of the first body 8.

In particular, the axial extremity of the first body 8 defining the first port 15, also defines the counter-stop 14 designed to rest on the mechanical stop 13 of the housing seat 12.

Preferably, the second port 16 is put in communication with the first port 15 thereby defining a single port extending between an axial extremity and a portion of the external side surface of the first body 8.

The side surface 11 also has a recess 17 defined in correspondence to an area substantially longitudinally aligned with the first protuberance 6a and substantially facing the second port 16. The area of the side surface 11 facing the second port 16 is therefore lowered with respect to the surrounding areas of the side surface itself.

The impact surfaces 7a and 7b of the protuberances 6a and 6b are suitably turned towards the first port 15. The first protuberance 6a is arranged upstream of the second protuberance 6b with respect to the direction of movement of the powder preparation P through channel 3. The direction is oriented from the inside of the channel itself towards the external opening 4.

Moreover, the first protuberance 6a is arranged on the opposite side to the second protuberance 6b and to the second port 16 with respect to the median plane of the channel 3.

The second body 9 then defines an air inlet mouth 18 communicating with the inhalation channel 3.

The inlet mouth 18 is placed in communication with the bottom wall of the housing seat 12 which defines the stop 13 and is therefore put in communication with the first port 15, which, in turn, is arranged in correspondence to the axial extremity of the first body 8 defining the counter-stop 14.

In the preferred embodiment of the inhalator 1, shown in FIGS. 1 to 4, the tank 5 has an insertion mouth 19 for the powder preparation P accessible from the outside and directly communicating with the tank itself. The insertion mouth 19 is therefore defined on the second body 9 in such a way that the tank 5 is accessible from the outside, whatever the position of the first body 8.

The inhalator 1 also includes a cover 20 associated in a removable way with the second body 9 and suitable for closing the insertion mouth 19.

Preferably, the inhalator 1 can be equipped with a plurality of covers 20 of different colors, each of which identifies a different type of medicinal product used in the inhalator.

In an alternative embodiment, shown in the figures from 5 to 7, the tank 5 is closed at the top, i.e., the second body 9 has a closing wall 23 defined integrally with the second body itself.

The second body 9 comprises an opening 24 facing the tank 5 and arranged on the opposite side with respect to the closing wall 23. Such opening 24 can be used to introduce the powder preparation into the tank 5 before the first body 8 is inserted into the housing seat 12.

The opening 24 communicates with the tank 5 when the first body 8 is removed from the housing seat 12, while it is isolated from the tank 5 when the first body 8 is inserted into the housing seat 12.

In both of the embodiments shown, the second body 9 has an ergonomic shape with two grip areas 21 on its outer surface that can be engaged by the user.

The grip areas 21 thus identify the grip of the inhalator 1 in a univocal way.

Advantageously, the inhalator 1 has identification means 25, 26 of the reciprocal position on the bodies 8 and 9 suitable for allowing blind users to easily determine the position of the first body 8 with respect to the second body 9.

More in detail, the identification means 25, 26 comprise a first fin 25 arranged on the outer surface of the first body 8 and a second fin 26 arranged on the outer surface of the second body 9 and arranged beside the tank 5.

The first fin 25 is therefore on the opposite side to the second fin 26 with the first body 8 in the first work, or operative, position, while it is substantially aligned with the second fin 26 when the first body 8 is in the second work, or operative, position.

The operation of the present invention is as follows.

After inserting the portion of the first body 8 comprising the ports 15 and 16 inside the housing seat 12, the first body itself is rotated to the first work position, so that its outer side surface closes the tank 5, isolating it from the channel 3.

Subsequently the cover 20 is removed from the second body 9 so that the tank 5 is accessible from outside for the insertion of the powder preparation P.

Once the powder preparation P has been introduced inside the tank 5, the latter is closed with the cover 20.

This situation is shown in FIG. 2, where the tank 5 contains a dose of powder preparation P and the first body 8 is in the first work position.

In the alternative embodiment of the inhalator according to the invention, shown in the figures from 5 to 7, the powder preparation must be introduced inside the tank 5 through the opening 24 before inserting the first body 8 in the housing seat 12. The introduction of the first body 8 in the housing seat 12, positioned so the second port 16 is in correspondence to the opening 24, maintains the powder preparation inside the tank 5.

This situation is shown in FIG. 6, where the tank 5 contains a dose of powder preparation P and the first body 8 is in the first work, or operative position.

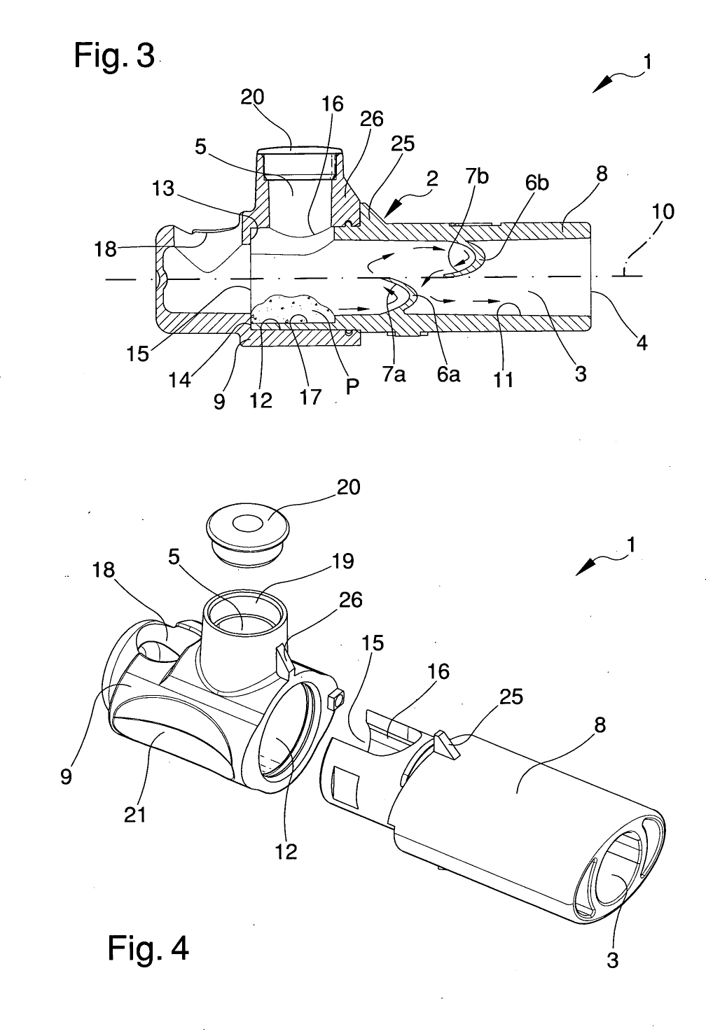

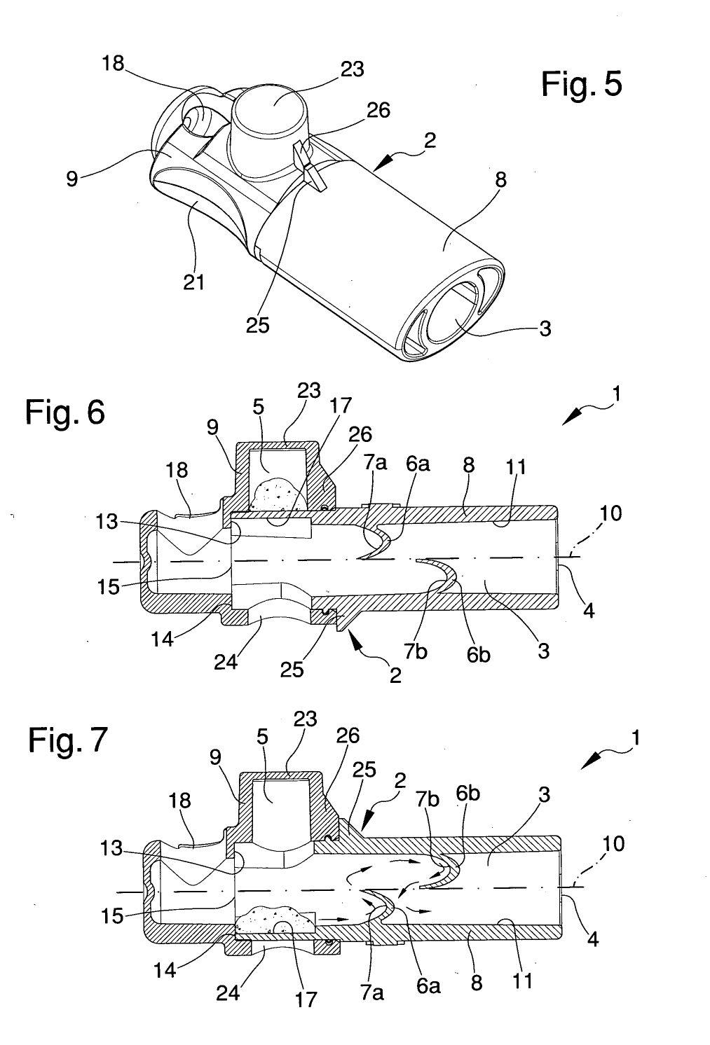

The user can therefore rotate the first body 8 with respect to the second body 9 by an angle substantially equal to 180°, bringing first body 8 to the second work position wherein the second port 16 is in correspondence to the tank 5. In this configuration, shown in the FIGS. 3 and 7, the tank 5 is placed in communication with the channel 3 and the powder preparation P falls by gravity inside the channel, resting on the side surface 11. More precisely, when the tank 5 is opened, the powder preparation P is collected up in correspondence to the recess 17.

The fall of the powder preparation can be observed by the user through the transparent walls of the first and of the second bodies 8 and 9.

At this point, the user introduces the external opening 4 inside his/her mouth and breathes in the air from the inlet mouth 18, thereby generating a flow of air inside the channel 3 directed towards the external opening 4.

This flow of air crossing the inhalation channel 3 reaches the powder preparation that has collected up inside the channel itself and drags this in its movement towards the external opening 4.

During the crossing of the channel 3 towards the external opening 4, the powder preparation P initially encounters the first impact surface 7a, which is substantially aligned longitudinally with the recess 17 on which the powder preparation itself collects up. Subsequently the powder preparation P encounters the second impact surface 7b, arranged downstream of the first protuberance itself with respect to the direction of movement of the powder preparation P inside the channel 3.

The C shape of the first and the second impact surfaces 7a and 7b gives the channel 3 a winding course to make the flow of air that conveys the powder preparation P towards the external opening 4 complete a substantially tortuous path identified by the arrows 22 shown in the FIGS. 3 and 7.

More in particular, the concave shape of the impact surfaces 7a and 7b is intended to cause substantial turbulence inside the channel 3, so as to increase the number of knocks or impacts of the particles of powder preparation P against the surfaces defined by the protuberances 6a and 6b and against the side surface 11 of the inhalation channel itself, as well as between the particles themselves.

It has, in point of fact, been ascertained that the described invention achieves the proposed objects. In particular the fact is underscored that the inhalator, according to the invention, obtains a more efficient inhalation with respect to the known inhalators, i.e., it substantially allows obtaining a high and effective breathable fraction.

More in particular, the concave shape characterising the impact surfaces defined by the protuberances of the inhalator results in the powder preparation having to complete a particularly tortuous path in its movement towards the mouth of the user and to cause a high number of knocks or impacts of the particles of the powder preparation against the walls delimiting the inhalation channel itself, and between the particles themselves.

The inhalator according to the invention, furthermore, allows the easy filling of the tank, and is therefore considerably more functional and practical than the inhalators of known type available on the market. Changes and revisions to applicant's inhalator can be achieved, without violating the spirit and scope of the invention. Consequently, the appended claims should be broadly construed in a manner consistent with the spirit and scope of applicant's invention, and should not be limited to their listed terms.

1. An inhalator for powder preparations, comprising a main body having:
an inhalation channel with an external opening accessible by a user;
a tank adapted to receive a dose of powder preparation that can be placed in communication with said inhalation channel;
a first body including said inhalation channel and a second body including said tank, said first body being rotatable with respect to said second body between at least a first position, in which said tank is isolated from said inhalation channel, and a second position, in which said first body places said tank in communication with said inhalation channel;
at least a protuberance protruding towards the inside of said inhalation channel and said protuberance defining at least a substantially concave impact surface, said impact surface intercepting said powder preparation during its travel through said inhalation channel towards said external opening,
wherein said impact surface being substantially C-shaped.

2. (canceled)

3. The inhalator according to the claim 1 wherein said at least one protuberance comprises two of said protuberances arranged spaced relative to each other along said inhalation channel and arranged on opposite sides with respect to a median plane passing through said inhalation channel.

4. (canceled)

5. The inhalator according to the claim 1, wherein said first body has an elongated shape, said inhalation channel extends along the longitudinal axis of said first body, and said at least one protuberance protrudes from the side surface of said inhalation channel crossways to said longitudinal axis of said inhalation channel.

6. The inhalator according to claim 3, wherein said protuberances are arranged spaced to each other along said longitudinal axis, said median plane is a longitudinal median plane of said inhalation channel.

7. The inhalator according to claim 1, wherein said second body comprises a housing seat or receiving a portion of said first body, said first body being rotatable with respect to said second body inside said housing seat.

8. The inhalator according to claim 7, wherein said first body is rotatable with respect to said second body around said longitudinal axis.

9. The inhalator according to claim 7, wherein said inhalation channel is of the through type and has a first port arranged on the side opposite to said external opening and said at least one protuberance a second port defined on the side surface of said first body, said first and said second ports being defined in the portion of said first body insertable into said housing seat, and said second port being suitable for establishing communication between said tank with said inhalation channel in said second position of the first body.

10. The inhalator according to claim 9, wherein said external opening and said first port are arranged in correspondence to the axial extremities of said first body, and said second port is substantially placed in between said external opening and said first port.

11. The inhalator according to claim 9, wherein said impact surface is turned towards said first port.

12. The inhalator according claim 9, wherein said first protuberance is arranged upstream of said second protuberance and on the opposite side to said second port with respect to said median plane.

13. The inhalator according to claim 1, wherein said second body includes an air inlet mouth communicating with said inhalation channel.

14. The inhalator according to claim 1, wherein said tank has an insertion mouth for inserting said powder preparation accessible from outside.

15. The inhalator according to the claim 14, wherein a cover is associated in a removable way with said second body and is suitable for closing said insertion mouth.
